(19) United States
(12) Patent Application Publication
(10) Pub. No.: US 2026/0263066 A1
(43) Pub. Date: Sep. 10, 2026

(54) ACTUATION DEVICE AND FEATURES FOR SURGICAL APPARATUSES

(71) Applicant: Arthrex, Inc., Naples, FL (US)

(72) Inventors: Joseph Aguila, Ft. Lauderdale, FL (US); Nathanael Gamso, Naples, FL (US); Derek C. Sullivan, Naples, FL (US)

(73) Assignee: Arthrex, Inc., Naples, FL (US)

(21) Appl. No.: 19/557,545

(22) Filed: Mar. 5, 2026

Related U.S. Application Data
(60) Provisional application No. 63/769,371, filed on Mar. 10, 2025.

(51) Int. Cl. A61B 17/062; A61B 17/04; A61B 17/29

(52) U.S. Cl. CPC A61B 17/062; A61B 17/0469; A61B 2017/2918; A61B 2017/2924

(57) ABSTRACT

An actuator for a surgical apparatus includes an elongated body extending along a longitudinal axis and an actuation structure. The actuation structure is operably coupled to the body and extends from a proximal end to a distal end. The actuation structure is configured to sequentially translate proximally and distally along the longitudinal axis.

### CROSS-REFERENCE TO RELATED APPLICATIONS

This application claims priority under 35 U.S.C. § 119 (e) and the benefit of U.S. Provisional Application No. 63/769,371 entitled ACTUATION DEVICE AND FEATURES FOR SURGICAL APPARATUSES, filed on Mar. 10, 2025, by Joseph Aguila, et al., the entire disclosure of which is incorporated herein by reference.

### BACKGROUND

The disclosure generally relates to an actuation device for a surgical apparatus and, more particularly, to an actuation device configured for single-handed use. The development of specialized devices for surgical routines continues to become increasingly important to support the corresponding development of advanced surgical procedures. While many improvements in the development of such products require increasingly complex assemblies, simple and intuitive operation of such devices is critical to their widespread utilization. As discussed in reference to various features in the following detailed description, the disclosure provides for improved features, assemblies, and corresponding methods for conducting surgical procedures that may be particularly applicable to complex endoscopic procedures.

### SUMMARY

In various implementations, the disclosure provides for an actuation device or actuator assembly for a surgical apparatus. In the primary examples demonstrated, the actuation device is illustrated as providing for a two-stage actuation control for operating a suture passer. However, the actuation device may be implemented for various surgical applications. An actuation structure of the actuation device may be coupled to an elongated body extending from a proximal end to a distal end. In operation, the actuation structure may be configured to sequentially translate proximally and distally to control sequential operations of an assembly of a surgical apparatus in which the actuation device is implemented.

In various implementations, the actuation structure of the actuation device provided by the disclosure may comprise a plurality of spring-biased arms that may interconnect proximal and distal end portions of an actuation structure. The proximal and distal ends of the plurality of arms may be connected to a proximal collar and a distal collar at opposing ends that, in operation, may extend proximally and distally in a prearranged sequence to facilitate the dual-stage actuation of the corresponding surgical apparatus. For example, in a retracted position, the plurality of arms may protrude outward from the elongated body with which the opposing collars are operably coupled. In response to a compressive force, the arms may be pressed inward toward the elongated body, resulting in longitudinal force applied to the opposing collars. A sequence or order of the translation of the opposing collars may be controlled by the relative stiffness of a first or proximal spring and a second or distal spring. By adjusting the stiffness and resulting spring force of each of the proximal and distal springs, the opposing force applied to the collars of the actuation device may be controlled to adjust a sequence of a proximal translation of the proximal collar and distal translation of the distal collar of the actuation structure. In this way, a sequence of a first stage and second stage of actuation of the surgical apparatus may be controlled by the associated spring pressure applied by the proximal and distal springs and the resulting order or sequence of translation of the corresponding proximal and distal collars.

While described primarily in relation to an exemplary suture passer, the actuation device may be implemented with a variety of surgical apparatuses. Additionally, the disclosure provides for a number of features and assemblies that may be implemented for a variety of surgical applications. For example, the needle assembly demonstrated in reference to the suture passer may provide for improved resiliency and maneuvering through various mechanical interfaces. The needle assembly may include a needle operably coupled to an elongated actuation rod. The needle may be interconnected with the elongated actuation rod via a flexible coupling. Further, a coil structure may be disposed over the flexible coupling, which may improve the wear response of the flexible coupling and provide a secondary connection between the needle and the elongated actuation rod to prevent detachment of the needle from the actuation rod. Accordingly, the disclosure provides for various features, assemblies, and associated methods of implementing the actuation structure, needle assembly, and suture passer to provide improved interaction and execution of a variety of surgical procedures.

These and other features, objects and advantages of the present disclosure will become apparent upon reading the following description thereof together with reference to the accompanying drawings.

### BRIEF DESCRIPTION OF THE DRAWINGS

FIG. 1 is a projected view of a surgical apparatus comprising an actuation device demonstrating the actuation of a suture passer in several configurations;

FIG. 2 is a side view of a surgical apparatus demonstrating a sequential activation of a plurality of stages of an actuation device;

FIG. 3A is a detailed schematic view of a clamping operation of a surgical apparatus controlled via an actuation device;

FIG. 3B is a detailed schematic view of a passing operation of a surgical apparatus controlled via an actuation device;

FIG. 4 is an exploded view of a suture passer assembly of a surgical apparatus;

FIG. 5A is a partially transparent projected view of an actuation device demonstrating an actuation assembly;

FIG. 5B is a partial assembly view of an elongated shaft and spring assemblies of the actuation device shown in FIG. 5A;

FIG. 6A is a side partial assembly view of a portion of an actuation device;

FIG. 6B is a detailed, partial assembly view of an actuation assembly demonstrating an exemplary coupling of a pushing member;

FIG. 7A is a side view of a handle assembly including a partial housing of an actuation device;

FIG. 7B is a partial assembly view of a receiving half of a portion of the handle assembly;

FIG. 8 is a detailed, partially transparent view of a suture passer assembly demonstrating details of a receiving mandible jaw;

FIG. 9A is a side schematic view of an actuation apparatus demonstrating various profile shapes of an actuation structure in accordance with the disclosure;

FIG. 9B is a side schematic view of an actuation apparatus demonstrating various profile shapes of an actuation structure in accordance with the disclosure; and

FIG. 9C is a side schematic view of an actuation apparatus demonstrating various profile shapes of an actuation structure in accordance with the disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

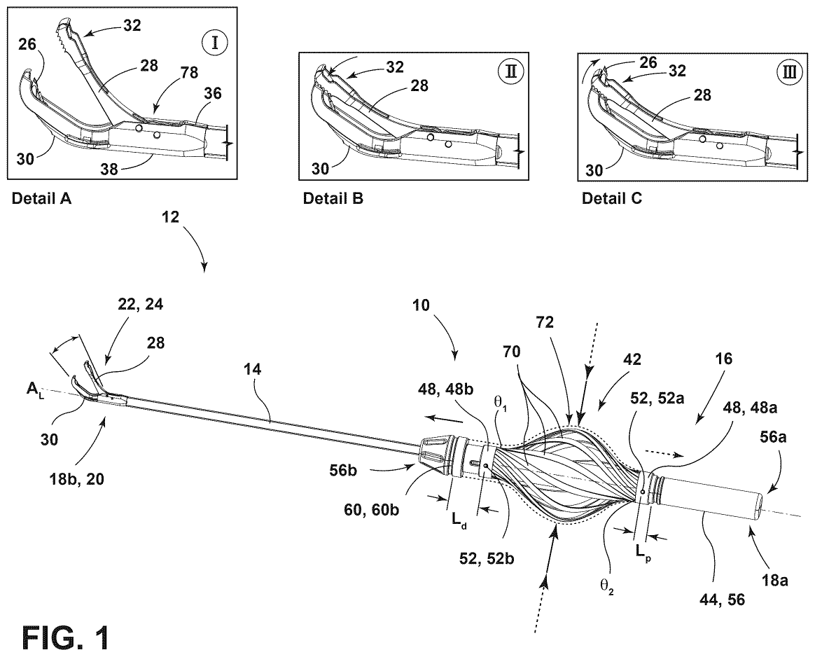

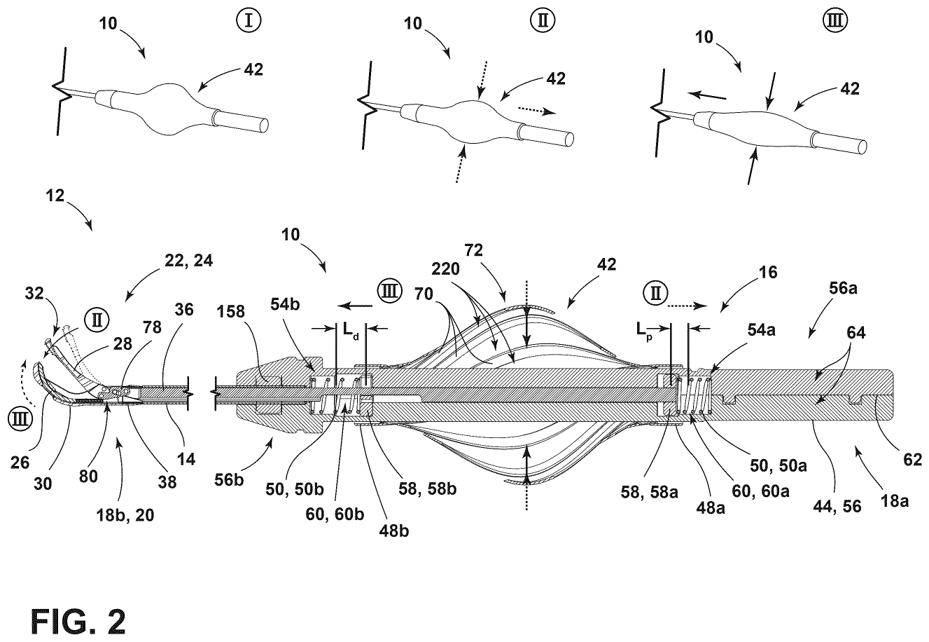

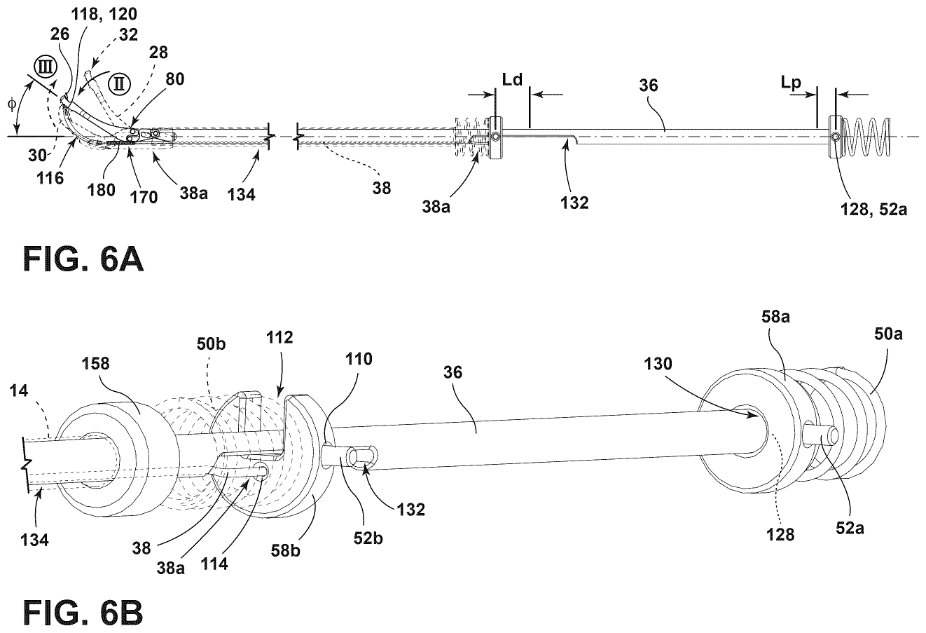

As generally depicted in FIGS. 1 and 2, the disclosure provides for an actuation device 10 and various features for a surgical apparatus 12. As demonstrated in various examples, the surgical apparatus 12 may correspond to an endoscopic surgical device comprising an elongated insertion shaft 14 or probe extending from a handle 16 at a proximal end portion 18a to an acting end 20 at a distal end portion 18b. The acting end 20 is demonstrated as a suture passing assembly 22 that may comprise a grasping assembly 24 configured to clamp tissue and pass a suture with a passing needle 26. As discussed in reference to various illustrated drawings throughout the disclosure, the actuation device 10 may provide for the sequential operation of two stages of the surgical apparatus 12. For example, a first stage of the actuation may include the closure of a mandible jaw 28 relative to a fixed jaw 30 of the grasping assembly 24. A second stage of the actuation may correspond to an extension of the passing needle 26 from the fixed jaw 30 and through a passing aperture 32 that may be formed through the mandible jaw 28. Though discussed in reference to the suture passing assembly 22, it shall be understood that the actuation device 10 may be implemented to control various surgical implements, particularly those that may be operated in multiple stages by the sequential operation of a pulling member 36 and a pushing member 38, as best demonstrated in FIG. 6 and later discussed. Accordingly, the disclosure may provide for the actuation device 10 and various features to improve the manipulation and operation of various surgical apparatuses.

As represented by the broken line in FIG. 1, the actuation device 10 may be enclosed by a cover 40, which may correspond to a flexible membrane or coating that may enclose an actuation structure. The flexible membrane or cover 40 may be of a polymeric, flexible textile, or similar materials and provide for a smooth or textured exterior surface enclosing or forming a bulb-like actuation structure 42. The actuation structure 42 may form a spring-biased assembly extending over a portion of an elongated body 44 comprising a handle portion 16 of the surgical apparatus 12. The bulb-like actuation structure 42 may be configured to compress, causing a proximal end portion 42a to extend sequentially with a distal end portion 42b. In the example shown, the proximal end portion 42a and the distal end portion 42b may correspond to a proximal collar 48a and a distal collar 48b, each of which may extend about and translate longitudinally along a portion of an elongated body 44. In this configuration, the translation of the proximal collar 48a may apply tension, drawing the pulling member 36 proximally toward the handle portion 16. Further, compression of the actuation structure 42 may cause the distal collar 48b to extend distally, translating the pushing member 38 toward the acting end 20.

As discussed in various examples in the following detailed description, the order of the sequence of the longitudinal translation of each of the collars 48a, 48b may be controlled by a spring force associated with a proximal spring 50a and a distal spring 50b that may oppose the motion of the proximal collar 48a and the distal collar 48b, respectively. In this way, the increased pressure or compression of the actuation structure 42 may be controlled to translate the proximal and distal end portions 42a, 42b and sequentially or concurrently pull and/or push the pulling member 36 and the pushing member 38. In the example shown, the sequence of operation of the surgical apparatus 12 may include:
I) a rest position wherein the actuation structure 42 is extended based on the spring-biased position of the actuation structure 42 interconnecting the proximal and distal end portions 42a, 42b;
II) a partially compressed configuration, wherein the mandible jaw 28 is closed relative to the fixed jaw 30 as a result of the proximal motion of the proximal collar 48a and the resulting pulling translating the pulling member 36 proximally toward the handle portion 16; and
III) a further compressed configuration of the actuation structure 42 resulting in the distal translation of the distal collar 48b, resulting in the pushing of the pushing member 38 distally, causing the pushing member 38 to extend the passing needle 26 through the passing aperture 32.

Though described as coil springs in the examples 50a, 50b; the sequential translation of the actuation structure 42 may be controlled via various biasing members.

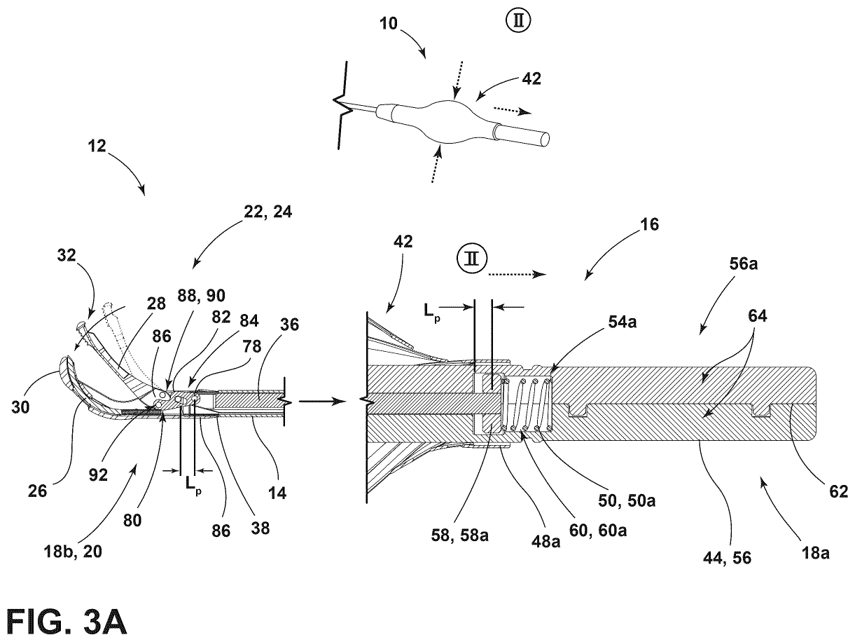

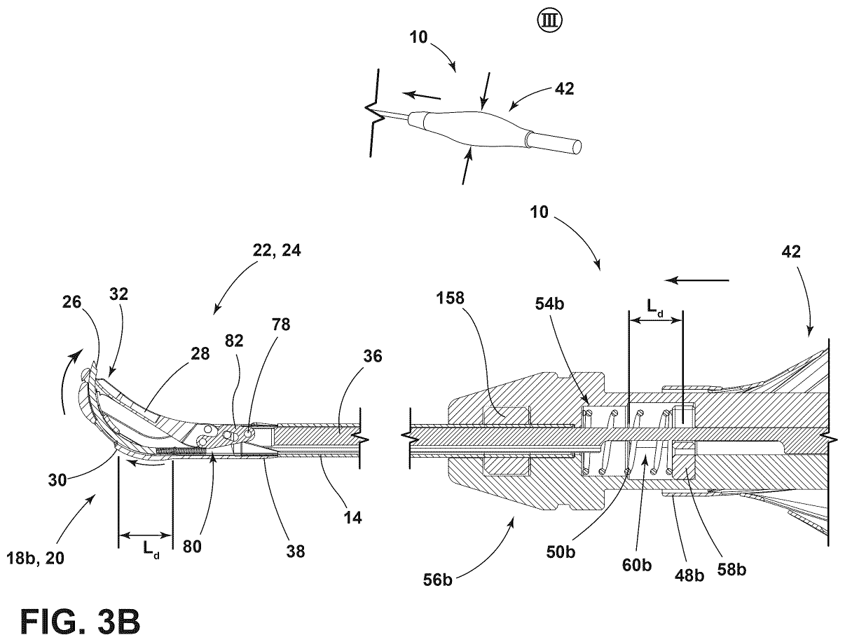

As shown in FIGS. 1-3, the exemplary operation of the actuation device 10 is demonstrated in reference to the exemplary implementation, including the sequential operation of the suture passing assembly 22. As demonstrated in FIG. 2 and further detailed in FIG. 3A, the first stage of compression of the actuation structure 42 resulting in the partially compressed configuration II of the actuation structure 42 may result in the proximal translation of the proximal collar 48a. As shown, the proximal collar 48a may extend about a generally cylindrical perimeter of the elongated body 44 and may engage a proximal pin 52a extending into a proximal pocket 54a formed by a housing 56 forming the elongated body 44. Within the proximal pocket 54a, a proximal spring 50a may be engaged to compress in response to the proximal translation of the proximal pin 52a engaging a proximal actuation sleeve 58a. The proximal translation of the proximal collar 48a, proximal pin 52a, and the proximal actuation sleeve 58a may be limited to extend through a proximal actuation slot 60a that may extend longitudinally through opposing sides of the housing 56.

As best demonstrated in FIG. 3A, the proximal actuation slot 60a may extend along a parting line 62 that may be formed by opposing sides of a clamshell assembly 64 of the housing 56. In this configuration, the proximal actuation slot 60 may extend along a proximal actuation length L_{P} relative to the housing 56. In this way, the engagement of the actuation structure 42 with the proximal actuation sleeve 58a and the proximal spring 50a may be controlled or restricted over the proximal actuation length L_{P}. As later discussed in reference to the sequential operation of the actuation device 10 and the related operation of the distal actuation spring 50b, the length of the proximal actuation slot 60a and a distal actuation slot 60b may define travel extents or limits of the proximal and distal end portions 42a, 42b of the actuation structure 42.

Referring now to FIGS. 2 and 3B, following the compression of the proximal spring 50a and corresponding translation of the proximal pin 52a over the proximal actuation length L_{P}, the proximal pin 52a may abut a proximal end portion of the proximal actuation slots 60a. Accordingly, the translation of the actuation structure 42 may be restricted from extending further proximally toward the handle portion 16. Accordingly, when additional pressure is applied compressing the actuation structure 42 as demonstrated in the further compressed configuration III, the distal end portion 42b of the actuation structure 42 may be forced to translate forward toward the distal end portion 18b of the surgical apparatus 12. Similar to the engagement of the proximal collar 48a, the distal collar 48b may engage a distal actuation sleeve 58b to compress the distal spring 50b by causing a distal pin 52b to extend longitudinally through a distal actuation slot 60b. As shown in FIG. 3B, the distal actuation slot 60b may extend over a distal actuation length L_{D}. In operation, the distal collar 48b may engage the pushing member 38, thereby translating the passing needle 26 distally through the insertion shaft 14. As a result, the passing needle 26 may extend through the passing aperture 32 formed in the mandible jaw 28 to pass a suture from the fixed jaw 30 to the mandible jaw 28.

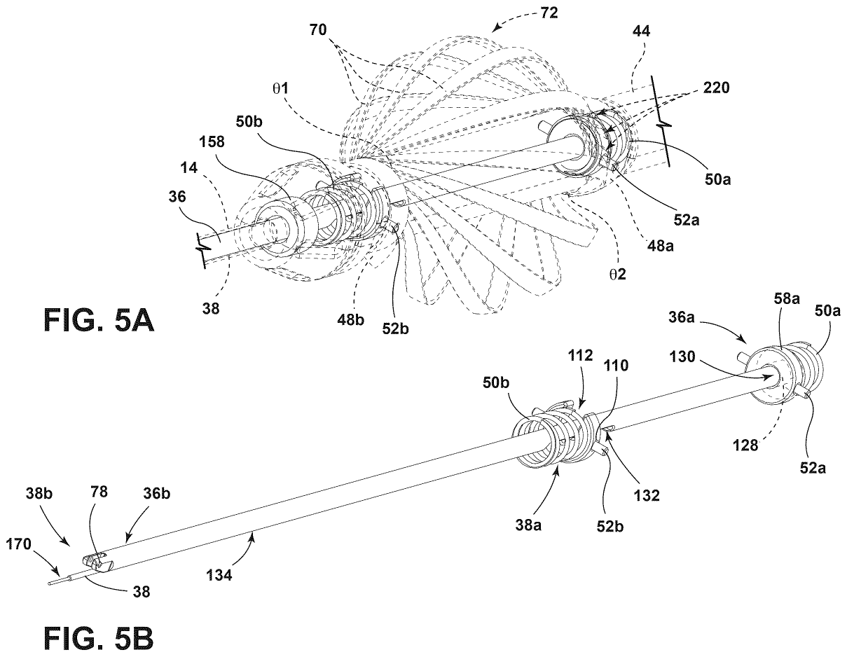

Still referring to FIGS. 1-3, the actuation structure 42 may comprise a plurality of arms 70 that may interconnect the proximal collar 48a to the distal collar 48b. In the example shown, the plurality of arms 70 are formed to have a spring-biased, bulbous profile shape 72 that extends outward from the elongated body 44 between the proximal end portion 42a and the distal end portion 42b. Each of the plurality of arms 70 may connect to the proximal and distal collars 48a, 48b at radially offset positions. For example, a first arm 70a may be operably coupled to the distal collar at a first radial position θ₁ extend along the bulbous profile shape 72 and interconnect with the proximal collar 48a at a second radial position θ₂. As shown, the second radial position θ₂ may be offset about the longitudinal axis A_{L} of the elongated body 44. Similar to the first arm 70a, each of the successive arms 70 (e.g., a second arm 70b, a third arm 70c, etc.) may interconnect with the distal collar 48b and the proximal collar 48a over a radial offset 40, similar to the offset between the first radial position θ₁ and the second radial position θ₂. See also FIG. 5A. In this way, each of the successive plurality of arms 70 extending radially about the longitudinal axis A_{L} may be interconnected with the proximal collar 48a and the distal collar 48b along a helical path 74. As shown, each of the plurality of arms 70 may correspond to slat-like beams that may have flattened rectangular cross sections. However, in various implementations, the plurality of arms 70 may be rectangular, round, ovular, arcuate, or include other shapes to form beam-like elements interconnecting proximal and distal collars 48a, 48b.

Referring still to FIGS. 1-3, the operation of the actuation device 10 may be configured to provide for the sequential actuation of the proximal end portion 42a and/or the distal end portion 42b of the actuation structure 42. As previously described, the bulbous profile shape 72 of the plurality of arms 70 may provide for a spring-biased configuration causing the arms 70 to deflect away from the elongated body 44 in a resting position that may be associated with the resting configuration I. As best illustrated in FIG. 2, the translation proximally associated with the partially compressed configuration II and distally associated with the further compressed configuration III may be controlled to occur sequentially in order or partially overlap based on the spring force associated with the proximal spring 50a and distal spring 50b and the corresponding reactive/opposing mechanical forces required to translate the pushing member 38 and the pulling member 36. In general, the sequential operation associated with the proximal translation of the proximal collar 48a and the distal translation of the distal collar 48b may be controlled by adjusting the stiffness and corresponding spring force of each of the proximal and distal springs 50a, 50b.

As later demonstrated symbolically for simplicity, the sequential operation of the actuation structure 42 is first described functionally. Beginning with the uncompressed configuration I, each of the proximal and distal springs 50a, 50b are demonstrated in FIG. 2 in extended positions. As the actuation structure 42 is compressed between the partially compressed configuration II and the further compressed configuration III, the pressure applied to the proximal and distal end portions 42a, 42b may increase correspondingly. As a result, the spring 50a, 50b with a lower spring force will compress first. Further, if the stiffness and corresponding spring force of one of the two springs 50a, 50b exceeds the spring force of the opposing spring compressed over the corresponding actuation length L_{P}, L_{D}; then the actuation associated with the spring 50a, 50b applying less force to the associated collar 48a, 48b will precede the translation of the opposing collar. In the specific example shown, the proximal spring 50a may apply a first spring force that may increase responsive to the compression of the actuation structure 42 and the translation of the proximal collar 48a toward the handle portion 16. The resulting spring force of the proximal spring 50a may reach a maximum when the proximal actuation pin 52a engages the proximal end of the proximal actuation slot 60a. As a result, the force opposing the longitudinal motion of the proximal collar 48a may no longer be associated with the spring force of the proximal spring 50a. Accordingly, the transition between the proximal actuation associated with the proximal collar 48a and the distal actuation associated with the distal collar 48b may be engaged responsive to the proximal pin 52a abutting the proximal end of the proximal actuation slots 60a. This transition may result in the shift from the partially compressed configuration II and proximal actuation of the proximal collar 48a to the further compressed configuration III of the actuation structure 42 and the corresponding distal translation of the distal collar 48b. As previously discussed, the order of the sequential translation proximally and distally may be adjusted based on the stiffness of the associated springs 50a, 50b as well as the lengths of the actuation slots 60.

$$\begin{matrix}Equation 1 & \\ If {F}_{P} = {K}_{P} \times {L}_{D} < {F}_{D} ( at rest ) , proximal then distal & ( Eq . 1 )\end{matrix}$$

Symbolically, the operation of the proximal spring 50a and the distal spring 50b may be more intuitive. As demonstrated in Equation 1, the spring force of the proximal spring is denoted as F_{P} and the spring force of the distal spring is denoted as F_{D}. Based on Hook's Law, depending on the spring constant, K_{P} for the proximal spring and KD for the distal spring, and the translation over the actuation lengths L_{P}, L_{D}, the resulting spring force of the proximal spring 50a and distal spring 50b may be configured to cause the sequential operation of the actuation structure 42 in any order. As shown in Equation 1, if the spring force F_{P} of the proximal spring 50a compressed over the proximal actuation length L_{P} is less than the distal spring force F_{D} at rest, the proximal collar 48a will translate before the distal collar 48b.

$$\begin{matrix}Equation 2 & \\ If {F}_{D} = {K}_{D} \times {L}_{D} < {F}_{P} ( at rest ) , distal then proximal & ( Eq . 2 )\end{matrix}$$

Alternatively, if the distal spring force F_{D} associated with the distal collar 48b extended distally along the distal actuation length L_{D} is less than the proximal spring force F_{P} at rest, then the distal spring 50b will compress before the proximal spring 50a (see Equation 2). As previously discussed, the difference in the proximal and distal spring forces F_{P}, F_{D} may be further distinguished based on the associated actuation lengths L_{P}, L_{D} of the actuation slots 60a, 60b. Accordingly, the actuation device 10 may provide for the sequential operation of multiple stages actuating the pulling member 36 and the pushing member 38 sequentially in a variety of ways depending on the desired configuration of the surgical apparatus 12.

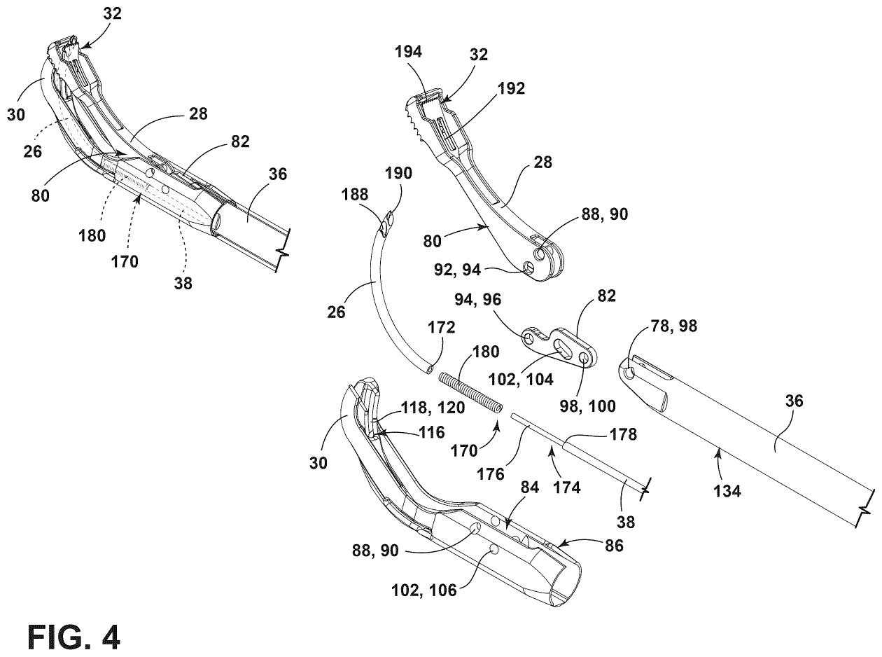

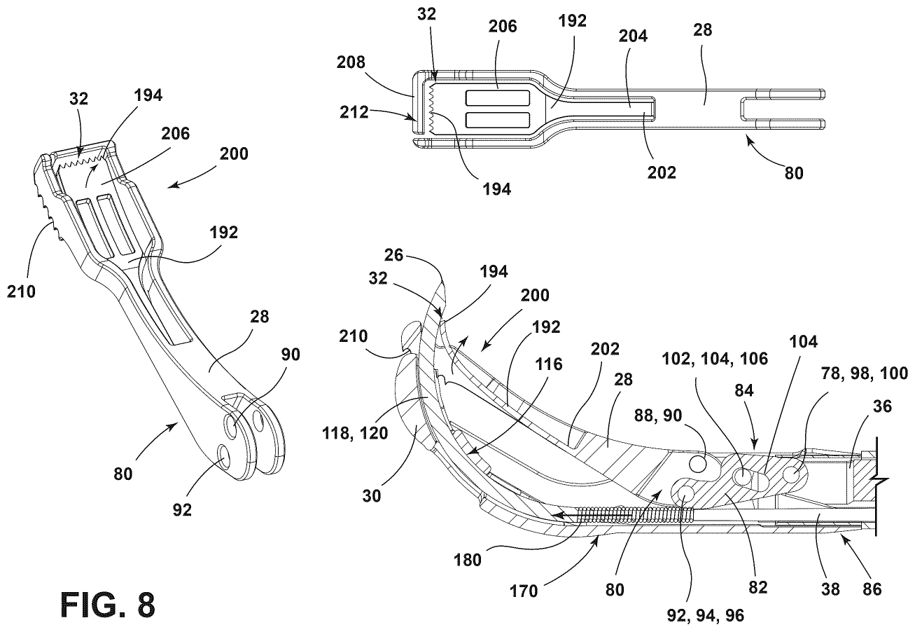

Referring now to FIGS. 4-6, the suture passing assembly 22 and the corresponding operation of the pulling member 36 and pushing member 38 are described in further detail. Referring first to FIG. 4, a detailed assembly view of the suture passing assembly 22 is shown as well as an exploded view for reference. As shown, the pulling member 36 may correspond to a pull rod and may form a distal coupling aperture 78 at the end portion operably coupled to a proximal pivoting interface 80 via an intermediate linkage 82. As best demonstrated in the side profile view of the suture passing assembly 22 in FIG. 8, the proximal pivoting interface 80 as well as the intermediate linkage 82 may be operably connected within a guide slot 84 formed within a proximal coupling portion 86 of the fixed jaw 30. For example, a pivoting pin 88 may interconnect a first aperture 90 of the proximal pivoting interface 80 to the opposing walls forming the guide slot 84 of the fixed jaw 30. Further, a second aperture 92 of the proximal pivoting interface 80 may receive a distal linkage pin 94 that may further engage a distal pivoting aperture 96 formed by the intermediate linkage 82. As illustrated in FIG. 4, the pin 94 as well as the other pins described are hidden to clearly demonstrate the corresponding structure of the grasping assembly 24. However, the pins are illustrated in the partially transparent side view depicted in FIG. 6A.

In operation, the intermediate linkage 82 may translate and rotate to accommodate the rotating path of the pivoting interface 80 of the mandible jaw 28 about the pivot pin 88. Accordingly, the intermediate linkage 82 may be operably coupled to the distal coupling aperture 78 of the pulling member 36 via a proximal pivoting aperture 98 and a proximal linkage pin 100. Finally, the intermediate linkage 82 may be operatively coupled to the fixed jaw 30 within the guide slot 84 via a central linkage pin 102 that may engage the elongated linkage slot 104 formed by the intermediate linkage 82. The central linkage pin 102 may further engage a second or intermediate aperture 106 formed through the fixed jaw 30 across the guide slot 84. In this configuration, proximal translation of the pulling member 36 may result in the pivoting and translation of the intermediate linkage 82 along the elongated linkage slot 104 and within the elongated guide slot 84. Further, the translation of the intermediate linkage 82 may cause the distal linkage pin 94 to pull the proximal pivoting interface 80 of the mandible jaw 28 via the second aperture 92, thereby rotating the mandible jaw 28 about the first or pivoting pin 88 to close the mandible jaw 28 relative to the fixed jaw 30.

Still referring to FIGS. 4-6, the operation of the actuation structure 42 in the further compressed configuration III may result in the distal translation of the distal collar 48b in connection with the distal pin 52b and the distal actuation sleeve 58b. As best illustrated in FIG. 6B, the distal actuation sleeve 58b may comprise a transverse mounting aperture 110 that interconnects the distal sleeve 58b to the distal collar 48b. Further, the distal actuation sleeve 58b may form a puck-like or flattened cylindrical form comprising a passing channel 112 through which a portion of the pulling member 36 may extend longitudinally along the longitudinal axis A_{L}. A mounting pocket may extend at least partially through a body of the distal actuation sleeve 58b proximate to a base or trough of the passing channel 112. In this configuration, a proximal end portion 38a of the pushing member 38 may engage the mounting pocket 114 or opening in the distal actuation sleeve 58b. As a result, the distal extension of the distal actuation sleeve 58b may result in the similar distal translation of the pushing member 38 along the longitudinal axis A_{L}. As demonstrated in FIG. 6A, the distal translation over the distal length L_{D} of the pushing member 38 may cause the pushing member 38 to extend the passing needle 26 through a distal guide channel 116 through the fixed jaw 30 and through the passing aperture 32 formed in the mandible jaw 28.

As further demonstrated in FIG. 6A, the distal guide channel 116 of the fixed jaw 30 may comprise a guide surface 118 forming a smooth guide profile 120 along which the passing needle 26 extends and deflects laterally away from the longitudinal axis A_{L} to pass between the fixed jaw 30 and the passing aperture 32 of the mandible jaw 28. As shown, the guide profile 120 formed by the guide surface 118 may correspond to an arcuate shape that may provide for the gradual transition of the passing needle 26 from within the guide channel 116 of the fixed jaw 30 outward through the passing aperture 32 formed in the mandible jaw 28 positioned proximately to the fixed jaw 30 in the closed configuration. In various implementations, the jaws 28, 30 of the passing assembly 22 may be angled at a grasping angle φ that may vary based on the specific application of the surgical apparatus 12. In the example shown, the grasping angle φ may be approximately 15° to 45° and, as shown in this specific example, may be approximately 25° to 35° relative to the longitudinal axis A_{L} of the insertion shaft 14.

In some implementations, the jaws 28, 30 and the associated the grasping angle ¢ may be adjustable or associated with an articulating assembly. For example, the jaws 28, 30 may be interconnected with the distal end of the insertion shaft via an articulating assembly that may allow the jaws to adjust in angle laterally (e.g., side-to-side, up-down) near the proximal coupling portion 86. The articulating assembly may correspond to one or more pull cables and/or gears that may be controlled via the controls included on the handle portion 16. Additionally, the jaws 28, 30 may be adjustable in various fixed positions of the grasping angle φ via one or more set screws to secure an interlocking angular assembly. In this way, the configuration of the actuation device 10 may be adjusted to suit various applications and user preferences.

Still referring to FIGS. 4-6, the pulling member 36 and the pushing member 38 may generally correspond to elongated rods that may be implemented to have primarily cylindrical cross-sectional shapes. However, the interaction between the pulling member 36 and the pushing member 38, while maintaining a minimum axis profile or diameter of the insertion shaft 14, may be achieved by implementing various features along the lengths of the members 36, 38. For example, beginning at a proximal end portion 36a of the pulling member 36, a proximal mounting aperture 128 may be configured to receive the proximal pin 52a and couple the pulling member 36 within a central opening 130 formed through the proximal actuation sleeve 58a. Continuing distally from the proximal actuation sleeve 58a as demonstrated in FIGS. 5 and 6, an elongated clearance slot 132 may form a partial cutaway portion of the cross section of the pulling member 36 extending along the longitudinal axis A_{L}. The elongated clearance slot 132 may provide for the pulling member 36 to extend through the passing channel 112 formed in the distal actuation sleeve 58b. In this configuration, the pulling member 36 may be free to translate along the longitudinal axis while allowing the distal pin 52b to freely pass through the clearance slot 132.

As previously discussed, the mounting pocket 114 of the pushing member 38 may be positioned proximal to the base of the passing channel 112. Accordingly, the pushing member 38 may be mounted parallel to the pulling member 36 and within or proximal to a transverse cross section of the pulling member. For example, the pushing member 38 may extend along the longitudinal axis within a diameter of the cylindrical structure forming the pulling member 36. The parallel alignment between the pulling member 36 and the pushing member 38 may extend from the proximal end portion 38a to the distal end portion 38b of the pushing member 38. To accommodate the length of the pushing member 38 along the corresponding portion of the pulling member 36, a narrow longitudinal passage or longitudinal channel 134 may be formed in a corresponding portion of the pulling member 36 extending from the elongated clearance slot 132 to a distal end portion 36b of the pulling member 36. In this configuration, the pushing member 38 may be translated proximally and distally through the longitudinal channel 134 responsive to the operation of the actuation device 10 without interfering with the operation of the pulling member 36. For clarity, the elongated clearance slot 132 and longitudinal channel 134, as well as the corresponding operation of the pulling member 36 and the pushing member 38, are best illustrated in FIGS. 5B and 6A of the appended figures.

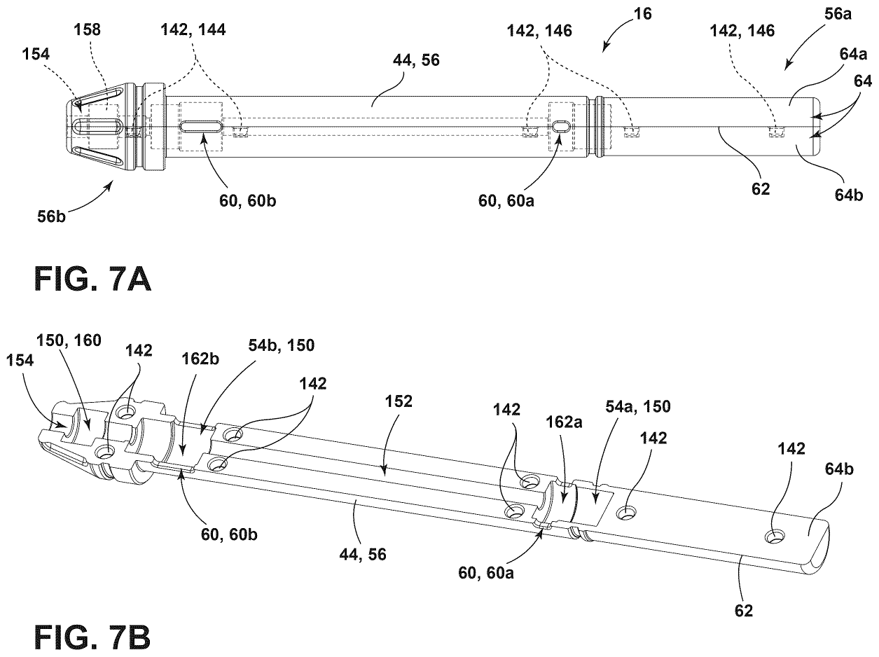

Referring now to FIGS. 2, 7A and 7B, the elongated body 44 formed by the housing 56 is described in further detail in reference to the operation of the pulling and pushing members 36, 38, respectively. As previously described, the housing 56 may comprise the clamshell assembly 64 including a first mating side 64a and a second mating side 64b. In various implementations, the mating sides 64a and 64b of the clamshell assembly 64 may be formed by molded plastic, diecast metal, or various other manufacturing techniques and may include a plurality of mounting features 142 that may support and/or house various components of the actuation device 10. Accordingly, the housing 56 and related mounting features 142 may provide for the actuation device 10 to be readily implemented and assembled to support a variety of surgical applications.

In the example shown, the first mating side 64a of the clamshell assembly 64 may correspond to a male connecting side and the second mating side 64b may correspond to a female receiving side. As demonstrated in FIG. 7B, the second mating side 64b may comprise a plurality of receiving apertures 144 configured to receive corresponding mounting protrusions 146 that may protrude from the first mating side 64a. In this configuration, the clamshell assembly 64 may be readily assembled and the opposing mating sides 64a, 64b may be aligned to house various components of the actuation device 10. In this way, the mounting features 142 of the housing 56 may ensure that the actuation device 10 and surgical apparatus 12 are readily manufactured without undue complexity and corresponding quality issues.

Still referring to FIGS. 7A and 7B, the housing 56 may form a plurality of interior pockets 150 that may generally be interconnected along a lumen or central passage 152 from the handle portion 16 at proximal end portion 56a to a shaft interface 154 at a distal end portion 56b. Beginning proximally, the interior pockets 150 of the mounting features 142 may include the proximal spring pocket 54a that may be interconnected with the distal spring pocket 54b via the central passage 152. As previously discussed, the pulling member 36 may extend through the central passage 152, through the distal spring pocket 54b, and further through the shaft interface 154 forming the distal end portion 56b of the housing 56. The pushing member 38 may extend from the distal actuation sleeve 58b positioned within the distal spring pocket 54b and outward through the shaft interface 154 to protrude from the distal end portion 56b of the housing 56.

As best demonstrated in the partially transparent view of the actuation device 10 in FIG. 5A, a shaft retention sleeve 158 may be disposed within a sleeve pocket 160 in the distal end portion 56b. In this way, a proximal end portion 14a of the insertion shaft 14 may be coupled to the shaft retention sleeve 158 and retained in connection with the housing 56 by securing the retention sleeve 158 in the sleeve pocket 160. Accordingly, the shaft interface 154 may provide for the insertion shaft 14 to be secured to the distal end portion 56b of the housing 56. Still referring to FIGS. 7A and 7B, the proximal and distal spring pockets 54a, 54b may be configured to receive and sized to accommodate the proportions of the proximal and distal springs 50a, 50b. Additionally, the spring pockets 54a, 54b may form a proximal cylindrical guide surface 162a and a distal cylindrical guide surface 162b through which cylindrical perimeter walls of the proximal actuation sleeve 58a and the distal actuation sleeve 58b may slide longitudinally along the proximal actuation length L_{P} and the distal actuation length L_{D}, respectively. Finally, extending from the cylindrical guide surfaces 162a, 162b, the proximal actuation slot 60a and the distal actuation slot 60b may be partially formed by each of the mating sides 64a, 64b of the clamshell assembly 64. In this way, the springs 50a, 50b; actuation sleeves 58a, 58b; and the pins 52a, 52b may be positioned within the proximal and distal spring pockets 54a, 54b and readily assembled in combination with the pulling member 36 and the pushing member 38 by connecting the mating sides 64a, 64b of the clamshell assembly 64.

Referring now to FIGS. 4 and 8, the suture passing assembly 22 is discussed in further detail in reference to the mandible jaw 28 and passing needle 26. As best demonstrated in FIG. 4, the passing needle 26 may connect to the distal end portion 38b of the pushing member 38 via a flexible coupling 170. More specifically, a proximal end portion 26a of the passing needle 26 may form a receiving pocket 172 configured to receive a narrow distal mating end portion of the pushing member 38. The mating end portion 174 may form a distal protrusion 176 extending from a transition shoulder 178 formed at the distal end portion 38b of the pushing member 38. Between the transition shoulder 178 and the engagement of the distal protrusion 176 within the receiving pocket 172 of the passing needle 26, a coil structure 180 may be interposed to reinforce the flexible coupling 170. In various implementations, the coil structure 180 may be fused, adhered, and/or welded at opposing end portions to the transition shoulder 178 and the proximal end portion 26a of the passing needle 26. In this configuration, the flexible coupling 170 between the passing needle 26 and the pushing member 38 may provide for increased flexibility relative to the body or cross section of the passing needle 26 while further reinforcing the interconnection between the passing needle 26 and the pushing member 38 with the interconnected coil structure 180 connected on opposing end portions of the flexible coupling 170. The configuration of the flexible coupling 170 may ensure that the assembly maintains increased flection while further providing additional mechanical connections between the pushing member 38 and the passing needle 26, which may prevent detachment between the pushing member 38 and the passing needle 26 throughout operation of the surgical apparatus 12.

As further demonstrated in FIGS. 4 and 8, the passing needle 26 and the mandible jaw 28 may include one or more features that may assist in passing a suture for various surgical procedures. At a distal end portion 26b, the passing needle 26 may comprise a suture retention notch 188 formed proximal of a narrow piercing tip 190. Upon extension from the distal guide channel 116 of the fixed jaw 30, the distal end portion 26b may translate through the passing aperture 32, thereby deflecting a retention tab 192. In the example shown, the retention tab 192 forms a cantilevered connection with a proximal portion of the perimeter of the passing aperture 32. In this way, an abrasive distal extent 194 of the retention tab 192 may engage and trap a suture protruding through the passing aperture 32. As best demonstrated in FIG. 8, the extension of the passing needle 26 responsive to the translation of the pushing member 38 may be sufficient to pass the suture retention notch 188 beyond the abrasive distal extent 194 of the retention tab 192. In this way, the retraction of the passing needle 26 may cause the abrasive distal extents 194 of the retention tab 192 to engage and trap the suture on a passing side 200 of the mandible jaw 28 opposite the fixed jaw 30.

Still referring to FIG. 8, the passing aperture 32 of the mandible jaw 28 may form a fork-like exterior profile shape configured to allow the retention tab 192 to freely deflect from a cantilevered proximal connection 202. Accordingly, the retention tab 192 may have an approximately uniform thickness extending from the cantilevered proximal connection 202 to the abrasive distal extent 194. Further, the width of the retention tab 192 may form an elongated profile shape comprising a narrow proximal portion 204 expanding outward to an expanded distal portion 206 that may terminate at the abrasive distal extent 194. In this configuration, the narrow proximal portion 204 may be configured to deform, providing for the deflection of the retention tab 192 while the expanded distal portion 206 may have an increased rigidity allowing the abrasive distal extent 194 to consistently catch and retain the suture between the abrasive distal extent 194 and a distal retaining wall 208 forming a distal extent of the passing aperture 32. In this way, the mandible jaw 28 may provide for a directional catch configured to pass the passing needle 26 in connection with a suture or similar textile structure or construct and retain a suture on the passing side 200.

Still referring to FIG. 8, in various implementations, the abrasive distal extent 194 may comprise a plurality of saw-like teeth that may serve to catch a material directionally passed within the suture retention notch 188 and retain the corresponding material on the passing side 200. Similar teeth 210 may also be incorporated at distal end portions of the mandible jaw 28 and the fixed jaw 30, which may ensure that the jaws 28, 30 effectively grasp tissue for interaction with the suture passing assembly 22. Once the suture or similar surgical construct is passed to the passing side 200, a surgeon may interact with a protruding portion and release the suture via a release slot 212 formed in the distal retaining wall 208. For example, due to the directional capture of the suture, a surgeon may need to continue pulling the suture distally through the passing side 200 and slide the material directionally toward the release slot 212 to remove the suture from the passing aperture 32.

Finally, as demonstrated in the representative figures, the mandible jaw 28 may be formed from various components and/or subassemblies. However, in the illustrated implementation, the mandible jaw 28 and the retention tab 192 may be formed from a common material having a monolithic structure, such as a molded or machined component. Such implementations of the mandible jaw 28 may include the cantilevered proximal connection 202, which may provide for improved manufacturability and consistency in operation of the surgical apparatus 12. Though discussed in reference to various exemplary structures, it shall be understood that the disclosed features and apparatuses may be implemented in various combinations while still providing the benefits discussed herein.

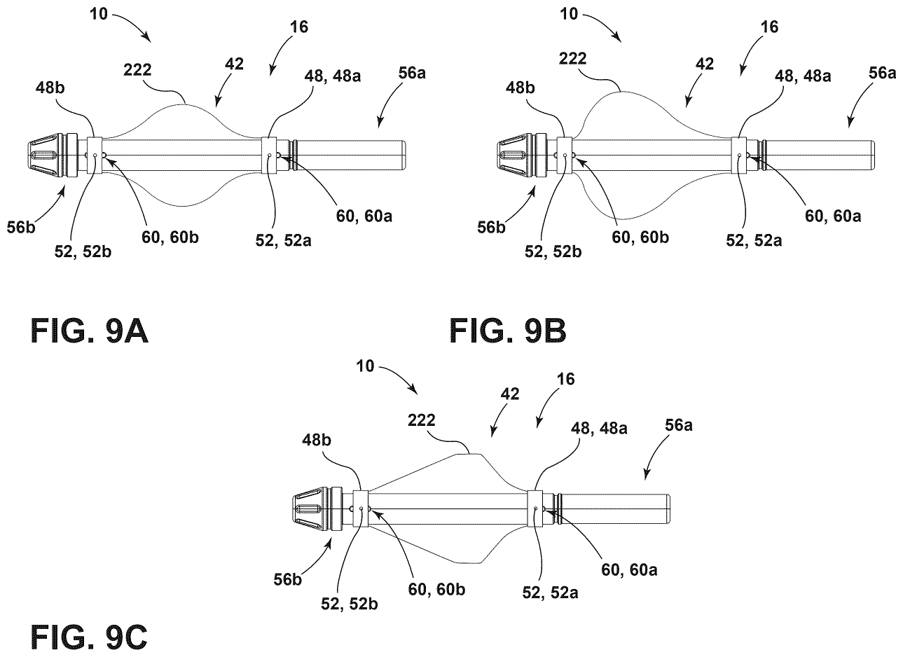

Referring now to FIGS. 9A-9C, various implementations of the actuation structure 42 are demonstrated showing examples of the bulbous profile shape 72 with the plurality of arms 70. As previously discussed, the plurality of arms 70 may correspond to spring-biased members interconnected on opposing sides by the collars 48a, 48b. More generally, the plurality of arms 70 and collars 48a, 48b may be formed from a polymeric or metallic cylindrical tube having a plurality of longitudinal slots 220 formed along a length defining and distinguishing the plurality of arms 70 (see FIG. 5A). In order to provide such a shape, the actuation structure 42 may be formed and/or heat treated to define the plurality of arms 70 in the bulbous profile shape 72 as described in various implementations herein.

In various implementations, the arms 70 and/or the collars may be formed from a thin-walled material having a thickness and width that may provide structural stability to maintain the profile shape 72 at rest while also limiting a spring force opposing the compression of the actuation structure from the resting state I to compressed states II and III. For example, depending on the bending modulus or flexural modulus of the material, the thickness (e.g., wall thickness) and/or width of the rectangular cross section of the plurality of arms 70 may vary. In some implementations, the thickness may vary from approximately 100-600 micron (4-24 thousandths of an inch or mils) and in implementations may vary from approximately 250-450 micron (10-18 mils) or from 300-400 micron (12-16 mils). A previously noted, the arms 70, or more generally the actuation structure 42, may be formed of various metallic materials (e.g., stainless steel, nitinol) or polymeric materials (polypropylene, polycarbonate) and may vary in thickness or width depending on the stiffness of the material and resistance to the deformation resulting from the actuation to the compressed states II, III.

In various implementations, the bulbous profile shape 72 may be adjusted to suit a preferred proportion for user engagement and/or grip style that may be associated with a specific surgical procedure. For example, as shown in FIG. 9A, the profile shape 72 may correspond to a smooth, symmetric wave pattern having a maximum amplitude positioned centrally along a length of the actuation structure 42. As shown in FIG. 9B, the profile shape 72 may have a similar smooth, wave-like profile however, at the maximum amplitude 222 of the profile shape 72, may be biased proximally as shown or distally toward the handle portion 16 in various implementations. Finally, as demonstrated in FIG. 9C, one or more portions of the profile shape 72 may correspond to angled or linear profiles that may extend between the proximal and distal end portions 42a, 42b of the actuation structure 42. In the specific example shown, the profile shape 72 may correspond to a wedge-like or triangular shape having the maximum amplitude 222 biased proximally toward the handle portion 16 of the surgical apparatus 12. Accordingly, the actuation structure may be implemented in a variety of ways to suit various surgical procedures or user preferences.

As discussed herein, the disclosure provides for various structures and features that may be implemented to provide an improved actuation device 10 that may be implemented for various surgical apparatuses. Further, various aspects of the exemplary suture passing assembly 22, including the jaws 28, 30, the passing needle 26, the passing aperture 32, and various other aspects, may be implemented in combination and/or independently depending on the required surgical application. Therefore, the disclosure provides for a wide variety of features and structures as well as corresponding methods for operation that may be implemented in various combinations to support a wide variety of surgical applications.

According to some aspects of the disclosure, an actuator for a surgical apparatus comprises an elongated body extending along a longitudinal axis and an actuation structure operably coupled to the body and extending from a proximal end to a distal end. The actuation structure is configured to sequentially translate proximally and distally along the longitudinal axis.

According to various aspects, the disclosure may implement one or more of the following features or configurations in various combinations:
the actuation structure comprises a plurality of arms interconnecting the proximal end and the distal end of the actuation structure;
the actuation structure comprises a first collar disposed at the proximal end portion and a second collar disposed at the distal end portion;
the first collar and the second collar are interconnected by the plurality of arms along the longitudinal axis;
each of the plurality of arms is connected to a proximal collar at a first radial position about the longitudinal axis and connected to a distal collar at a second radial position about the longitudinal axis;
the second radial position is radially offset from the first radial position about the longitudinal axis;
each of the plurality of arms extends helically about the longitudinal axis between a proximal collar and a distal collar operably connected to the elongated body;
the plurality of arms comprise beam elements extending along a length from a proximal collar to a distal collar operably connected to the elongated body;
the length of the beam elements extends away from the longitudinal axis along a profile shape;
the beam elements form a cross-sectional shape along the length, the cross section being one of a rectangular, round, oval, or arcuate shape;
the profile shape extends away from the longitudinal axis in a retracted position of the actuation structure;
the proximal collar is in a first rest position and the distal collar is in a second rest position in the retracted position of the actuation structure;
the profile shape comprises at least one of a rounded, bulb-like profile and a trapezoidal profile extending radially outward from the longitudinal axis between the proximal collar and the distal collar;
the sequential translation proximally and distally is in response to an applied force to at least a portion of a circumferential compression surface formed by the actuation structure about the longitudinal axis.
the actuation structure is a component of an actuation assembly that comprises a first spring and a second spring disposed in the elongated body;
a difference in a spring stiffness of the first spring relative to the second spring controls an order of the sequential translation of the actuation structure proximally and distally along the longitudinal axis;
the proximal end portion of the actuation structure is operably coupled to the elongated body via a proximal slot formed through the elongated body along the longitudinal axis over a first length;
the distal end portion of the actuation structure is operably coupled to the elongated body via a distal slot formed through the elongated body along the longitudinal axis over a second length;
the first length is different from the second length;
the proximal translation of the actuation structure extends along a first length and the distal translation extends along a second length different from the first length;
a first biasing member disposed in the elongated body proximal the first slot, wherein the first biasing member has a first stiffness;
a second biasing member disposed in the elongated body proximal the second slot;
the second biasing member has a second stiffness different from the first stiffness;
the first stiffness is less than the second stiffness;
a first spring force of the first biasing member compressed over the first length is less than or equal to a second spring force of the second biasing member in an extended position;
the proximal collar and the distal collar are operably coupled to the body via a proximal pin and a distal pins that engage the proximal slot and the distal slot, respectively; and/or
a compression of the first pin is opposed by the first biasing member and restricted to the first length of the proximal slot.

According to another aspect of the disclosure, a suture passer comprises an elongated probe extending from a distal end portion of the elongated body and a jaw assembly including opposing jaws. A first jaw of the opposing jaws is a configured to open and close relative to a second jaw in response to a first actuation of the sequential translation of an actuation structure.

According to various aspects, the disclosure may implement one or more of the following features or configurations in various combinations:
the actuation structure comprises the actuating assembly previously described;
a passing needle configured to extend from the second jaw and through a passing aperture of the first jaw in response to a second actuation responsive to the sequential translation of the actuation structure;
a pulling member extending through the elongated probe and operably coupled to the first jaw;
the first actuation is a proximal translation of the actuation structure that retracts the pulling member proximally;
the retraction of the pulling member closes the first jaw relative to the second jaw;
a pushing member extending through the elongated probe and coupled to the passing needle;
the second actuation is a distal translation of the actuation structure that extends the pushing member distally;
the extension of the pushing member extends the passing needle from the second jaw and through the passing aperture of the first jaw;
n. the pulling member extends through a channel formed through at least a portion of the pulling member;
the first jaw further comprises a retention tab extending into the passing aperture that deflects away from the second jaw in response to the extension of the passing needle through the passing aperture;
the retention tab is cantilevered from a proximal perimeter of the passing aperture formed by a mandible body of the first jaw;
the retention tab forms a flexible beam extending to a distal tab end comprising grooves configured to capture a suture between the distal tab end and a distal perimeter wall of the passing aperture; and/or
first jaw and the retention tab form a monolithic structure formed by a common material.

According to yet another aspect of the disclosure, a passing assembly for a suture passer comprises a needle including a proximal connecting end portion and a distal piercing end portion. An elongated actuation rod is operably coupled to the proximal connecting end portion via a flexible coupling. A coil structure is disposed over the flexible coupling. The flexible coupling is coupled to the needle and the elongated actuation rod.

According to various aspects, the disclosure may implement one or more of the following features or configurations in various combinations:
the passing assembly is in incorporated in the suture passer;
an elongated actuation rod extends from a proximal end portion to a distal end portion and the distal end portion comprises a narrow protrusion extending from an elongated body of the elongated actuation rod;
a shoulder is formed about the narrow protrusion where the narrow protrusion extends from the elongated body;
the coil structure is disposed over the narrow protrusion and is connected to the elongated body proximal to the shoulder at a first coil end;
the coil structure is connected to the proximal connecting end portion of the needle at a second coil end, opposite the first coil end; and/or
the proximal connecting end portion of the needle forms a receiving aperture that receives the distal end portion of the narrow protrusion.

According to another aspect of the disclosure, a surgical apparatus comprises an elongated body extending along a longitudinal axis; an actuation structure operably coupled to the body and extending from a proximal end to a distal end, wherein the actuation structure is configured to sequentially translate in a first actuation; and a second actuation, wherein the sequential transition extends a proximal translation and a distal translation along the longitudinal axis. A suture passer comprises an elongated probe extending from a distal end portion of the elongated body; a jaw assembly comprising opposing jaws; and a passing needle. A first jaw of the opposing jaws is a configured to open and close relative to a second jaw in response to the first actuation. The passing needle is configured to extend from the second jaw and through a passing aperture of the first jaw in response to the second actuation.

According to various aspects, the disclosure may implement one or more of the following features or configurations in various combinations:
the opening and closing of the first jaw relative to the second jaw is responsive to the proximal translation; and/or
the extension of the passing needle through the passing aperture is responsive to the distal translation.

As used herein, words of approximation such as, without limitation, “approximately,” “substantially,” or “about” refer to a condition that when so modified is understood to not necessarily be absolute or perfect but would be considered close enough to those of ordinary skill in the art to warrant designating the condition as being present. Further, the lack of such modifying terms does not otherwise require strict interpretation of the corresponding value or property. Instead, the extent to which the associated interpretation varies will depend on how great a change can be instituted and still have one of ordinary skill in the art recognize the modified feature as having the required characteristics or capabilities of the unmodified feature. Such determinations may vary considerably depending on the technological field based on the corresponding equivalency associated with the described operation or property. In general, but subject to the preceding discussion, a numerical value herein that is modified by a word of approximation such as “approximately” may vary from the stated value by ±0.5%, ±1%, ±2%, ±3%, ±4%, ±5%, ±10%, ±12%, or ±15%.

Any element in a claim that does not explicitly state “means” for performing a specified function or “step” for performing a specified function, should not be interpreted as a “means” or “step” clause as specified in 35 U.S.C. § 112.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. An actuator for a surgical apparatus comprising:
an elongated body extending along a longitudinal axis; and
an actuation structure operably coupled to the body and extending from a proximal end to a distal end, wherein the actuation structure is configured to sequentially translate proximally and distally along the longitudinal axis.

2. The actuator according to claim 1, wherein the actuation structure comprises a plurality of arms interconnecting the proximal end and the distal end of the actuation structure.

3. The actuator according to claim 2, wherein the actuation structure comprises a first collar disposed at the proximal end portion and a second collar disposed at the distal end portion.

4. The actuator according to claim 3, wherein the first collar and the second collar are interconnected by the plurality of arms along the longitudinal axis.

5. The actuator according to claim 2, wherein each of the plurality of arms is connected to a proximal collar at a first radial position about the longitudinal axis and connected to a distal collar at a second radial position about the longitudinal axis.

6. The actuator according to claim 5, wherein the second radial position is radially offset from the first radial position about the longitudinal axis.

7. The actuator according to claim 2, wherein each of the plurality of arms extends helically about the longitudinal axis between a proximal collar and a distal collar operably connected to the elongated body.

8. The actuator according to claim 1, wherein the sequential translation proximally and distally is in response to an applied force to at least a portion of a circumferential compression surface formed by the actuation structure about the longitudinal axis.

9. The actuator according to claim 1, wherein the actuation structure is a component of an actuation assembly that comprises a first spring and a second spring disposed in the elongated body.

10. The actuator according to claim 9, wherein a difference in a spring stiffness of the first spring relative to the second spring controls an order of the sequential translation of the actuation structure proximally and distally along the longitudinal axis.

11. The actuator according to claim 1, wherein the proximal end portion of the actuation structure is operably coupled to the elongated body via a proximal slot formed through the elongated body along the longitudinal axis over a first length.

12. The actuator according to claim 11, wherein the distal end portion of the actuation structure is operably coupled to the elongated body via a distal slot formed through the elongated body along the longitudinal axis over a second length different from the second length.

13. A suture passer comprising:
an elongated probe extending from a distal end portion of the elongated body; and
a jaw assembly comprising opposing jaws, wherein a first jaw of the opposing jaws is a configured to open and close relative to a second jaw in response to a first actuation of a sequential translation of an actuation structure.

14. The suture passer according to claim 13, further comprising:
an actuating assembly comprising:
an elongated body extending along a longitudinal axis; and
an actuation structure operably coupled to the body and extending from a proximal end to a distal end, wherein the actuation structure translates in over a first length proximally and a second length distally in the sequential translation.

15. The suture passer according to claim 13, further comprising a passing needle, wherein the passing needle is configured to extend from the second jaw and through a passing aperture of the first jaw in response to a second actuation responsive to the sequential translation of the actuation structure.

16. The suture passer according to claim 15, further comprising:
a pulling member extending through the elongated probe and operably coupled to the first jaw.

17. The suture passer according to claim 16, wherein the retraction of the pulling member closes the first jaw relative to the second jaw.

18. The suture passer according to claim 16, further comprising:
a pushing member extending through the elongated probe and coupled to the passing needle.

19. The suture passer according to claim 18, wherein the second actuation is a distal translation of the actuation structure that extends the pushing member distally.

20. A surgical apparatus comprising:
an elongated body extending along a longitudinal axis;
an actuation structure operably coupled to the body and extending from a proximal end to a distal end, wherein the actuation structure is configured to sequentially translate in a first actuation and a second actuation, wherein the sequential transition extends a proximal translation and a distal translation along the longitudinal axis; and
a suture passer comprising:
an elongated probe extending from a distal end portion of the elongated body;
a jaw assembly comprising opposing jaws;
a passing needle; and
wherein a first jaw of the opposing jaws is a configured to open and close relative to a second jaw in response to the first actuation, and the passing needle is configured to extend from the second jaw and through a passing aperture of the first jaw in response to the second actuation.
